(19) United States
(12) Patent Application Publication
(10) Pub. No.: US 2002/0151583 A1
(43) Pub. Date: Oct. 17, 2002

(54) Prevention of liver cancer by administration of simvastatin

(76) Inventor: Assa Weinberg, Los Angeles, CA (US)

Correspondence Address: Dave B. Koo, Esq., Squire, Sanders & Dempsey L.L.P., 801 S. Figueroa Street, 14th Floor, Los Angeles, CA, 90017-5554, US

(21) Appl. No.: 09/832,380

(22) Filed: Apr. 11, 2001

(51) Int. Cl. A61K 31/366; G01N 33/53

(52) U.S. Cl. 514/460; 435/7.1

(57) ABSTRACT

A method for preventing liver cancer is disclosed. In one embodiment, the method comprises administrating an effective amount of simvastatin to patients showing elevated alphafetoprotein levels. In another embodiment, the method comprises administrating simvastatin to patients upon detection of a liver condition, i.e viral Hepatitis B, Hepatitis C and liver cirrhosis.

### BACKGROUND OF THE INVENTION

In spite of substantial advances in the understanding of the mechanism of oncogenesis (transformation of a normal cell into a malignant one), a method to prevent and reverse such oncogenic process has not been put into practice. Many environmental, dietary and genetic factors play a significant role in the formation of a malignant tumor, but no drug is known to prevent the formation of a malignant tumor. In many cases, patients affected with a malignant tumor are deemed to be terminal.

Cancer of the liver or hepatocellular carcinoma is a particular case in point. It is an aggressive and rapidly spreading disease. Even with the combined use of the four classic treatment modalities, -surgery, radiation, chemotherapy and immunotherapy—little has been changed to alter the grim prospect. The only methods capable of eradicating liver cancer at the present time are partial resection of the liver in the case where the malignancy is localized or complete extirpation of the malignant liver and a replacement by transplantation. In liver transplantation cases, the cost of the procedure is substantial, followed by a lifelong administration of anti-rejection drugs, and medical follow-up involving doctor visits, blood tests, and imaging studies.

Patients with advanced liver diseases, and in particular, patients with viral hepatitis B and C are known to be at a high risk of developing liver cancer. In the United States, there are 70,000 new hepatitis B cases and more than 250,000 newly diagnosed hepatitis C cases each year. Around the world and especially in Southeast Asia and China, Hepatitis B and C are at epidemic proportion without a cure in sight. The case is particularly serious as the Hepatitis virus can be transmitted during delivery to the newborn baby. This was demonstrated in recent studies from Taiwan in which 40% of babies born to Hepatitis B or C carrier mothers were found to be positive for the same, a few days after delivery or a month later. In the same token hepatocellular carcinoma is at epidemic proportions in this region and represents a major public health issue for these countries.

By monitoring the levels of a tumor marker, an early detection of the formation of a malignant liver tumor mass is possible. Yet, with no drug or other medical procedures available to prevent the formation of liver cancer, these patients even with an early detection are left to no meaningful medical treatment until they develop detectable malignant tumor masses.

A drug that could prevent the formation of hepatocellular carcinoma would be a major contribution not only for the patients in question, but would also bring significant financial and general public health benefits.

### SUMMARY OF THE INVENTION

The invention described herein is a novel method of preventing the formation of liver cancer. The method according to the present invention comprises the oral administration of an effective dose of a drug having a previously unknown anti-cancer property.

The method described herein is particularly useful in preventing the formation of liver cancer in patients with advanced liver diseases, i.e. viral hepatitis B and C.

### DESCRIPTION OF THE INVENTION

Simvastatin is butanoic acid, 2,2-dimethyl-,1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1α,3α,7β,8β(2S*,4S*),-8aβ]]. The empirical formula of simvastatin is C₂₅H₃₈O₅ and its molecular weight is 418.57. Its structural formula is:

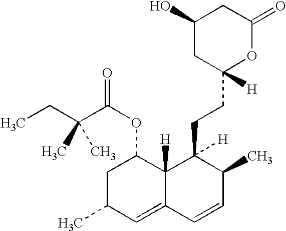

Simvastatin is a known cholesterol-lowering agent that is derived synthetically from the fermentation of Aspergellus Ferrous. Simvastatin has been used extensively in the treatment of hyperlipidemia and its pharmacology and toxicology are well known.

Use of simvastatin in the prevention of malignant diseases, especially liver cancer or hepatocellular carcinoma, has been previously unknown. According to the present invention, by administering simvastatin to patients with an existing liver condition, and by monitoring the level of alphafetoprotein, a liver tumor marker, the formation of liver cancer can be prevented.

The detection of liver cancer was facilitated when oncologists identified a tumor marker, alphafetoprotein, whose level is elevated in the serum of patients with hepatocellular carcinoma. Subsequent studies have demonstrated that the elevation of alphafetoprotein precedes, by several months to a year, the detection of liver mass by physical examination or imaging studies. An elevated alphafetoprotein level is thus now considered to be an advance notice for the liver specialist that a malignant process is about to appear, especially if the level is very high. When there is an elevated level of alphafetoprotein, it is assumed that the malignant process has started, but involves a number of cells so small that they cannot be detected by current imaging technologies. A method of prevention of liver cancer should therefore be able to lower and maintain the level of alphafetoprotein over a prolonged period of time.

Administration of simvastatin according to the present invention has shown to both lower and maintain the tumor marker alphafetoprotein at a normal level, thereby indicating its usefulness in preventing the formation of liver cancer. The use of simvastatin in the prevention of a malignant liver tumor according to the present invention contemplates the daily administration of an effective amount of simvastatin orally. An effective amount of simvastatin is that amount which provides a decrease in the level of alphafetoprotein to a normal level and which maintains alphafetoprotein at the normal level.

Patients who underwent this treatment with an elevated alphafetoprotein level have seen their level progressively decline over several weeks to several months to normal values. The alphafetoprotein level is considered normal at or below 10 nanograms/mL and such alphafetoprotein level above 10 nanograms/mL (ng/mL) is considered elevated. None of the patients subsequently developed liver cancer.

Additionally, administration of simvastatin not only normalizes elevated alphafetoprotein levels, but also prevents the elevation of such levels in people who are at a high risk of developing a liver tumor. This was seen in the group of patients with known liver diseases. While these patients' liver diseases continued to progress, especially those with viral hepatitis, no malignancy was ever detected by careful imaging studies and no elevation of alphafetoprotein was detected. It is therefore shown that simvastatin is an agent that is able to prevent the formation of the malignant process prior to its occurrence. It is also shown that simvastatin can reverse oncogenesis on a cellular level even after the process was initiated, but prior to the formation of a detectable malignant tumor mass.

Clinical Case Reports:

In order to avoid unforeseen side effects, the treatment was initiated at a 20 mg dose once daily for one week. The dose was then increased to 40 mg daily during the following week, and was again increased to 60 mg daily during the third week. During the fourth week, the dose was increased to 80 mg daily (two tablets of 40 mg each) and remained the same thereafter.

The effective amount of simvastatin to prevent the formation of liver cancer according to the present invention is not limited to the dosages described above, but may range from 10 mg to 500 mg per day depending on various factors such as the body weight of patients and the level of alphafetoprotein.

Following the administration of simvastatin as described above, the patients' alphafetoprotein levels were monitored on a monthly basis until values reached normal levels and then every three months thereafter in association with liver function tests and coagulation studies as part of a follow-up. In addition, an abdominal ultrasound test was performed every four months in an attempt to identify any new liver masses, and MRI (magnetic resonance imaging) studies of the liver were performed.

Because of the ethical considerations, treatments were offered only to terminally ill patients with a disease status considered by their oncology experts to be hopeless.

Case No. 1:

When first seen, a 72-year-old retired pathologist with a known Hepatitis B infection for the last 30 years was clinically asymptomatic with an elevated liver function test and prolonged prothrombin time with an INR (international normalized ratio) of 1.9 indicating advanced liver disease. The patient's alphafetoprotein level was rapidly increasing over the last several months, and the patient was suspected of developing liver cancer, although imaging studies of her liver detected no masses. The patient was then placed on progressively increased dosages of simvastatin and her alphafetoprotein level normalized over the next four months. Her alphafetoprotein level remained normal. Subsequently, on recommendation by another physician, the patient stopped taking simvastatin. Her alphafetoprotein level rose to 33, and simvastatin was re-introduced at progressively increased dosages. Again, her alphafetoprotein level normalized and has remained normalized. Repeated imaging studies of her liver never showed any liver tumor mass.

Case No. 2

A 53 years old newspaper editor with a known Hepatitis B infection for the last 27 years was first seen for an assessment of her Hepatitis B for which she was placed on anti-retroviral drugs with remarkable results. In spite of the clinical success, she developed progressively increasing levels of alphafetoprotein, which was confirmed by multiple laboratory tests in and out of the United States. She was placed on increasing doses of simvastatin. Her alphafetoprotein level normalized within 5 months and remained normalized with maintenance dosages of simvastatin. Repeated imaging studies of her liver never showed any liver tumor mass.

Case No. 3

A 46 year-old woman with a known Hepatitis C for the last 20 years was found to have an elevated alphafetoprotein level on a routine follow-up. She initially refused to take simvastatin even though the consulted second opinion informed her that there were no known preventive measures to the formation of liver cancer. Her alphafetoprotein level continued to increase, and when it reached above 50, she returned and asked to be placed on the drug. Following the protocol administration of simvastatin, her alphafetoprotein level normalized within 2 months and remained normalized. Repeated imaging studies of her liver never showed any liver tumor mass.

Case No. 4

These cases involve the administration of simvastatin to patients with known Hepatitis B and C, and other causes of liver cirrhosis, mainly alcoholic. All of these patients had normal alphafetoprotein levels and were given simvastatin on a daily basis since their first diagnosis of their liver disease. None of these patients showed an elevation of alphafetoprotein during an observation period. The specificity of simvastatin toward the prevention of a malignant process can be drawn from the fact that the liver function test of these patients continued to deteriorate in spite of the administration of simvastatin, but no elevation of alphafetoprotein occurred.

Even though no direct experiments were conducted to establish the cellular mechanism by which simvastatin caused the progressive regression of elevated alphafetoprotein levels of the above mentioned cases, the similarity in the response in all of these cases, and the progressive mode of the improvements demonstrate the therapeutic effect of simvastatin to prevent and reverse the formation of liver cancer in patients with an elevated alphafetoprotein level. The cases also show that simvastatin is useful in preventing the formation of liver cancer in patients with known liver diseases who are at a significant risk for developing liver cancer. In all of the patients studied, the clinical improvement was easily obtained without any dramatic event and the improvement was progressive and long lasting. In all of these cases, the alphafetoprotein levels normalized and stayed normalized over an extended period of time.

While the foregoing description defines an embodiment of the present invention, it is to be understood that it is subject to many modifications and changes without departing from the spirit and scope of the appended claims.

1. A method for preventing the formation of liver cancer, comprising:
monitoring alphafetoprotein levels to detect an increase in the alphafetoprotein level; and
administering an effective amount of simvastatin upon detection of an elevated level of alphafetoprotein.

2. The method according to claim 1, wherein said effective amount of simvastatin is the effective amount necessary to lower the alphafetoprotein level.

3. The method according to claim 2, wherein said simvastatin is introduced when the alphafetoprotein elevated level is above 10 ng/mL, and wherein the normal level is at and below 10 ng/mL.

4. The method according to claim 1, wherein said effective amount of simvastatin is a dosage between 20 mg to 80 mg per day.

5. The method according to claim 1, wherein said effective amount of simvastatin is a dosage between 10 mg to 500 mg per day.

6. A method for helping to prevent the formation of liver cancer, comprising:
monitoring alphafetoprotein levels; and
administering an effective amount of simvastatin to patients with a liver condition, wherein said effective amount is effective to maintain a normal alphafetoprotein level.

7. The method according to claim 6, wherein said simvastatin is introduced upon the detection of said liver condition selected from the group consisting of viral Hepatitis B, Hepatitis C and liver cirrhosis from alcohol, idiopathic and genetic causes.

8. A method for preventing the formation of liver cancer, comprising:
monitoring the level of a liver tumor marker; and
administering an effective amount of simvastatin upon detection of an elevated level of the tumor marker.
